(19) United States
(12) Patent Application Publication
(10) Pub. No.: US 2019/0274869 A1
(43) Pub. Date: Sep. 12, 2019

(54) Vacuum Prosthetic Penis, System and New Method of Intercourse

(71) Applicant: Perfect Fit Brand, Inc., Wilton Manors, FL (US)

(72) Inventor: Stephen Callow, Los Angeles, CA (US)

(21) Appl. No.: 16/293,761

(22) Filed: Mar. 6, 2019

Related U.S. Application Data
(60) Provisional application No. 62/639,473, filed on Mar. 6, 2018.

(51) Int. Cl. A61F 5/41

(52) U.S. Cl. CPC A61F 5/41; A61F 2005/414; A61F 2005/412; A61F 2005/411

(57) ABSTRACT

A sexual aid device, system and method comprising: an exterior wall; interior air bladder; flange; an outlet; penile sheath; penile port; wherein air is removed from the interior air bladder causing the penile sheath to expand toward the exterior wall and adhere to and engorge an inserted penis. In an optional saddled embodiment the inner and outer scrotal support of the chassis and sheath help to hold the device in place while providing a comfortable scrotal opening an enabling gentle massage of the scrotum and perineum through use of a perineum bump. In order to remove air from the interior air bladder a bulb, piston, motorized and/or other vacuum pumps may be used.

### CROSS-REFERENCE TO RELATED APPLICATIONS

This application claims the benefit of U.S. Provisional Pat. Appl. No. 62/639,473, filed Mar. 6, 2018 and entitled Vacuum Prosthetic Penis, System and New Method of Intercourse. This invention improves upon prior inventions of the inventor. See, e.g., Stephen Callow, Unified Dildo and Bottom Open Anatomically Contoured Harness Plate, U.S. Appl. Pub. No. US20160279019A1 filed Mar. 26, 2015; Stephen Callow, Personal cleaning device U.S. Appl. Pub. No. US20090082729A1, filed Sep. 26, 2007; Stephen Callow, Hollow Plug, U.S. Pat. No. D742025, issued Oct. 27, 2015; and Stephen Callow, Hollow dildo, U.S. Pat. No. D719,665 issued Dec. 16, 2014, each of which are incorporated herein by reference in their entirety.

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### STATEMENT REGARDING FEDERALLY SPONSORED RESEARCH OR DEVELOPMENT

Not Applicable

### THE NAMES OF PARTIES TO A JOINT RESEARCH AGREEMENT

Not Applicable

### INCORPORATION-BY-REFERENCE OF MATERIAL SUBMITTED ON A COMPACT DISC

Not Applicable

### BACKGROUND OF THE INVENTION

### Field of the Invention

The invention relates to the fields of sexual health devices, systems and methods for sexual intercourse. The invention provides a solution for erectile dysfunction. The invention enables sexual intercourse with a flaccid penis and also enables sexual intercourse to begin or continue after climax. The invention enables those unable to maintain an erection on their own to have the necessary support for penetration while maximizing the donning user's sensation and intimacy. The invention also provides a form of attachment directly to the donning users penis that does not require an erection and is an improvement over straps and harnesses.

### Description of the Related Art

Erectile dysfunction is treated in a variety of manners. Common treatments include drugs such as: Avanafil (Stendra); Sildenafil (Viagra); Tadalafil (Adcirca, Cialis); Vardenafil (Levitra, Staxyn) Generally, these drugs enhance the effects of nitric oxide and natural chemicals in the body that relax muscles in the penis and/or increase blood flow in the penis. Generally, taking one of these drugs will not automatically produce an erection, but enables the user to form an erection once the drug is active and nitric oxide is released.

Other drugs such as alprstadil can be administered through self-injection. With this method the user or their partner injects alprostadil (caverject impuls, edex, etc.) into the base or side of the penis. Other drugs such as paverine and pentolamine might also be used alone or in conjunction (bimix/trimix) with alprostadil. In some cases, these drugs may cause more immediate erections than oral medications. However, there can be pain and bleeding at the injection site, formation of fibrous tissue, nerve damage, infection and in some cases prolonged erection (priapism). Alternative methods of application of the drugs include urethral suppositories. These suppositories and the tools that insert them can be painful and the overall treatment suffers from many of the same problems as with other drugs.

Other possible side effects of treating ED with drugs include flushing, nasal congestion, headache, visual changes, backache and stomach upset. In some cases allergic reactions, sever pain, death and suicide may result. Medications for erectile dysfunction do not work in all men and might be less effective in certain conditions, such as after prostate surgery or if the persons afflicted have diabetes. Some medications might also be dangerous if the afflicted persons: take nitrate drugs such as: isosorbide mononitrate (Monoket) and isosorbide dinitrate (Dilatrate-SR, Isordil) nitroglycerin (Minitran, Nitro-Dur, Nitrostat, etc) and others. Drugs may also be dangerous for those that have heart disease, heart failure and low blood pressure (hypotension). In addition or in lieu of drugs, afflicted persons may be treated with hormones such as testosterone replacement. This can have many of the same side effects as drugs and also cause other changes in the body and mood of the user.

Instead of and/or to supplement drugs and hormones, afflicted persons may use herbal supplements and alternative medicine. Some of these can be dangerous and may have unknown methods of work and may interact with prescription drugs or be contaminated during formulation.

Generally, change of lifestyle and exercise has shown to improve erectile function in some men, such as quitting smoking, abstaining from alcohol and recreational drug use, losing excess pounds, reducing stress and psychological therapy. Such treatments take time and discipline and might not always be effective or appropriate.

In lieu or in addition to other therapies, surgery and penile implants are sometimes a solution. This form of treatment typically involves surgically placing devices into the penis. The devices may include inflatable or bendable rods. The inflatable devices enable the user some control how long they have an erection whereas the bendable rods help the penis be firm and malleable. Surgery is risky, generally permanent and can have many side effects, injuries and drawbacks including but not limited to permanent pain, greater dysfunction and or infection.

Yet, another alternative is the use of vacuum construction devices (also known as penis pumps or “VCD”), and erectile rings. Generally, such devices are used by placing a tube over the afflicted penis. Then the pump removes air from inside the tube. This creates a vacuum that pulls blood into the penis causing an erection. A tension ring is then placed around the base of the penis to restrict the blood from leaving and keep the penis firm. The penis pump may damage or bruise the penis, and ejaculation is often restricted by the tension ring. The use of a VCD can also result in a colder feeling penis than one that has become erect naturally. The inventor has created many devices that can be helpful for extended sexual intercourse and use by those that suffer from erectile dysfunction or premature ejaculation. However, these inventions do not provide the unique benefits for better fit, form, method of use and manufacture, particularly when used with a flaccid penis.

There is a long unmet need for a device that does not rely upon drugs or restricting blood flow from the penis that can be used on the fly that is comfortable and erotic for use and that promotes sexual wellness. Moreover, although the inventor has created many devices that can are helpful for extended sexual intercourse and use by those that suffer from erectile dysfunction or premature ejaculation, but none of those inventions provide the unique structure disclosed herein that fills long felt needs for better fit, form, new methods of use and superior manufacture, particularly when used with a flaccid penis. See, e.g., Stephen Callow, Unified Dildo and Bottom Open Anatomically Contoured Harness Plate, U.S. Appl. Pub. No. US20160279019A1 filed Mar. 26, 2015; Stephen Callow, Personal cleaning device U.S. Appl. Pub. No. US20090082729A1, filed Sep. 26, 2007; Stephen Callow, Hollow Plug, U.S. Pat. No. D742, 025, issued Oct. 27, 2015; and Stephen Callow Hollow dildo, U.S. Pat. No. D719,665 issued Dec. 16, 2014, each incorporated herein by reference in their entirety.

### SUMMARY OF THE INVENTION

The present invention is a vacuum prosthetic penis, system and new method of intercourse.

More specifically, the vacuum prosthetic penis, system and new method of intercourse enables intercourse for person with a flaccid penis due to erectile dysfunction, sexual exhaustion (post climax). It also provides enhanced sexual experience for persons with normal and above normal sexual drive and ability.

With the invention of the vacuum prosthetic penis, system and new method of intercourse disclosed herein now sexual partners can try previously avoided positions with new confidence, comfort and pleasure. The invention enables deeper penetration for longer times, and is easy to don by a user with a flaccid or exhausted penis. It does not require straps or a harness. It provides the donning user with greater support and confidence and aids in increasing sexual stamina, sexual performance, stronger erections, and sexual pleasure.

Although the invention is illustrated and described herein as embodied as a sexual aid device, system and encore method of intercourse, the invention is not limited to the details shown because various modifications and structural changes may be made without departing from the invention and the equivalents of the claims. However, the construction and method of operation of the invention together with additional objects and advantages thereof will be best understood from the following description of specific embodiments when read in connection with the accompanying drawings.

### BRIEF DESCRIPTION OF THE DRAWINGS

Features and advantages of the present invention will become apparent to those skilled in the art from the following description with reference to the drawings, in which:

FIG. 1 is an elevated side view of the present invention with an outlet towards the base of the shaft;

FIG. 2 is an elevated side view of the present invention with an outlet towards the tip of the head;

FIG. 3 is an elevated side view of the present invention with an outlet towards the base of the shaft with a connected pump bulb;

FIG. 4 is top down view of the configuration shown in FIG. 1;

FIG. 5 is top down view of the configuration shown in FIG. 2;

FIG. 6 is top down view of the configuration shown in FIG. 3;

FIG. 7 a cutaway side elevated side view of the invention of FIG. 1;

FIG. 7 is a cutaway side view of the invention of FIG. 1 with an inserted penis;

FIG. 9 is a cutaway side view of the invention of FIG. 2 with a bulb shaped pump located in the head;

FIG. 10 show the invention of FIG. 1 with an external pump mechanism connected to the outlet towards the base;

FIG. 11 is a top down view of the configuration of FIG. 10.

FIG. 12 is side view of the configuration of FIG. 10 invention as donned by a user with a soft penis;

FIG. 13 is side view of the configuration of FIG. 10 invention as donned by a user after a vacuum pump has removed air from the internal air bladder;

FIG. 14 is side view of the invention centering focus on the penile chamber;

FIG. 15 is an example of one of many textures that are applied to the interior of the penile chamber;

FIG. 16 is an example of one of many textures that are applied to the interior of the penile chamber;

FIG. 17 is an example of one of many textures that are applied to the interior of the penile chamber;

FIG. 18 is an example of one of many textures that are applied to the interior of the penile chamber; and,

FIG. 19 is an example of one of many textures that are applied to the interior of the penile chamber.

### DETAILED DESCRIPTION OF THE INVENTION

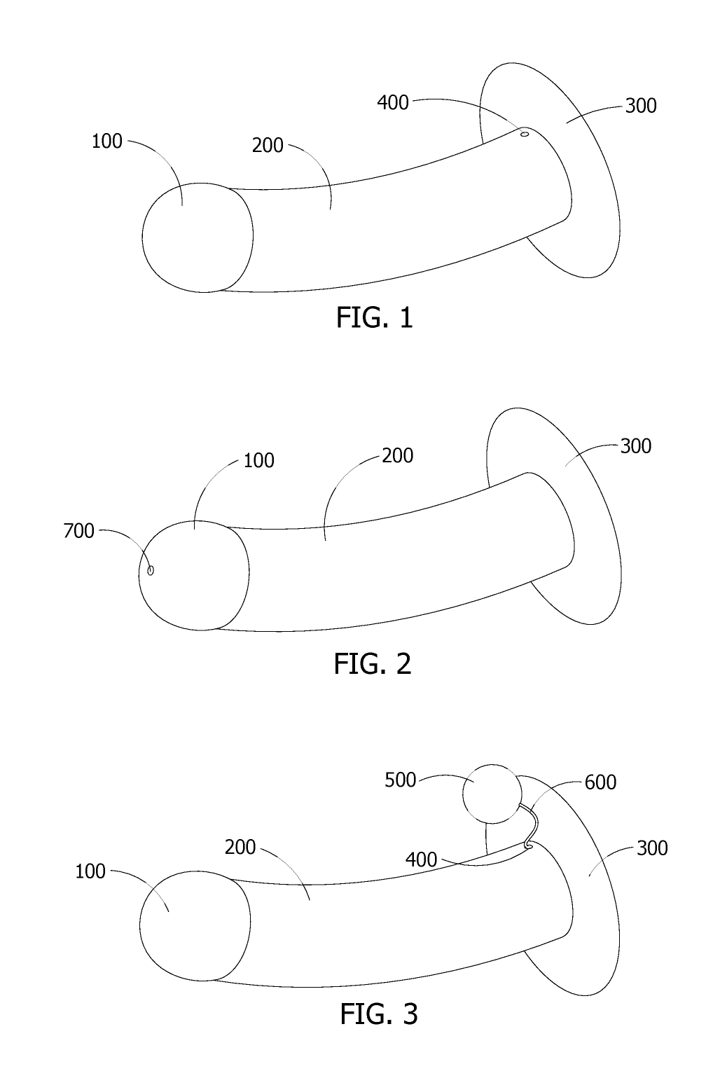

Referring to the drawings FIG. 1 is an elevated side view of the present invention with an outlet towards the base of the shaft. Shown in FIG. 1. are the: head 100; shaft 200; flange 300; and, base shaft outlet 400.

FIG. 2 is an elevated side view of the present invention with an outlet towards the tip of the head. Shown in FIG. 2. are the: head 100; shaft 200; flange 300; and, head tip outlet 700.

FIG. 3 is an elevated side view of the present invention with an outlet towards the base of the shaft with a connected pump bulb. Shown in FIG. 3. are the: head 100; shaft 200; flange 300; base shaft outlet 400; base shaft pump connector 600; and, exterior base shaft pump 500.

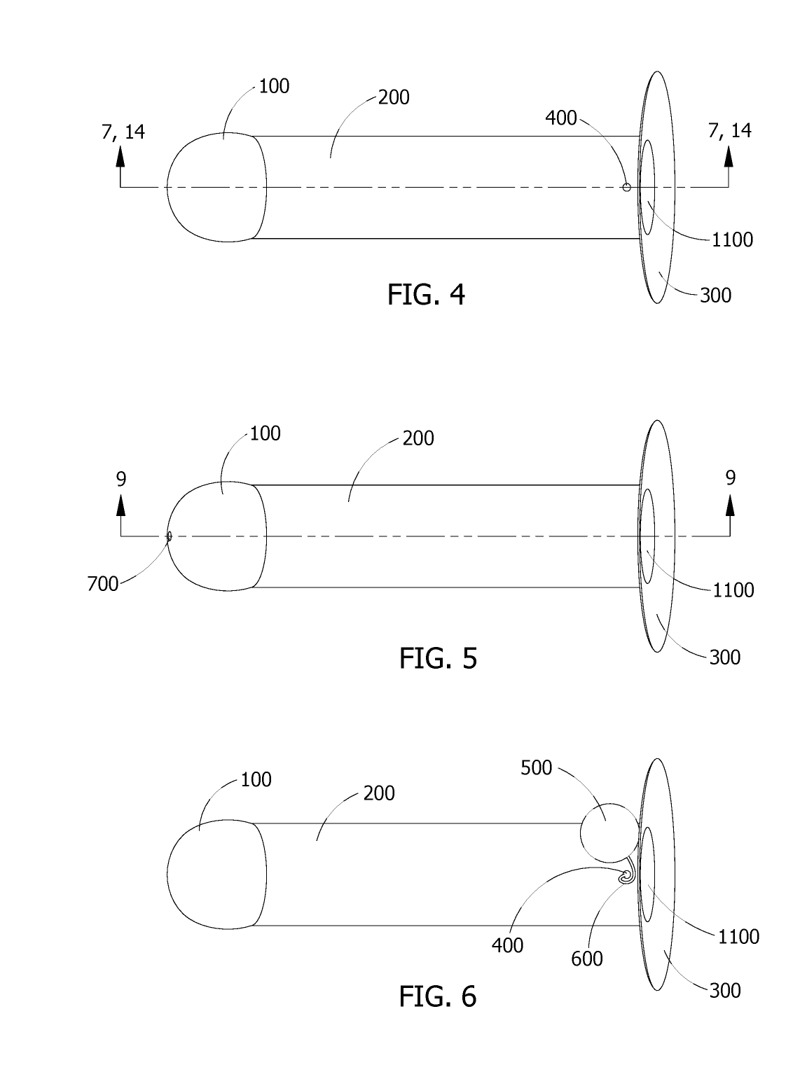

FIG. 4 is top down view of the configuration shown in FIG. 1. Shown in FIG. 4 are the: head 100; shaft 200; flange 300; base shaft outlet 400; and, penile port 1100.

FIG. 5 is top down view of the configuration shown in FIG. 2. Shown in FIG. 5 are the: head 100; shaft 200; flange 300; head tip outlet 700; and, penile port 1100.

FIG. 6 is top down view of the configuration shown in FIG. 3. Shown in FIG. 6 are the: head 100; shaft 200; flange 300; base tip outlet 400; pump connector 600; exterior base shaft pump 500; and, penile port 1100.

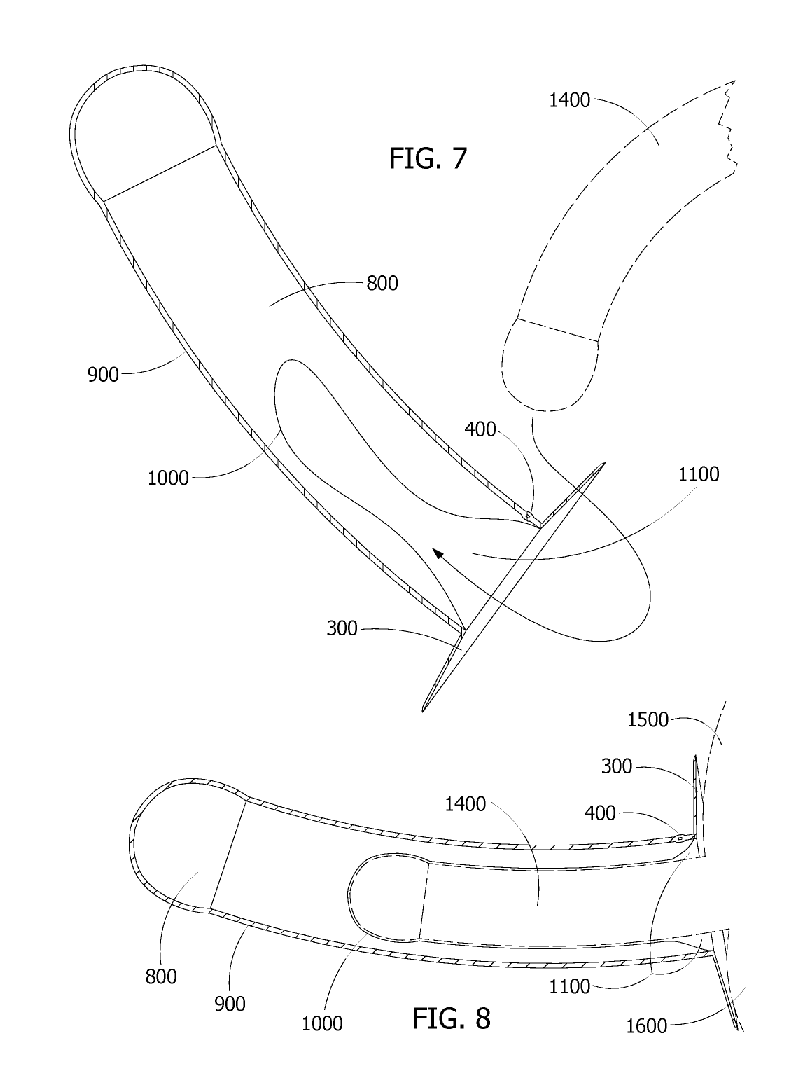

FIG. 7 a side elevated side view of the invention of FIG. 1. Shown in FIG. 7 are the: exterior wall 900; interior air bladder 800; flange 300; base shaft outlet 400; penile sheath 1000; penis 1400; and, penile port 1100.

FIG. 8 is a cutaway side view of the invention of FIG. 1 with an inserted penis. Shown in FIG. 8 are the: exterior wall 900; interior air bladder 800; flange 300; base shaft outlet 400; penile sheath 1000; penile port 1100; penis 1400; user abdomen 1500; and, user scrotum 1600.

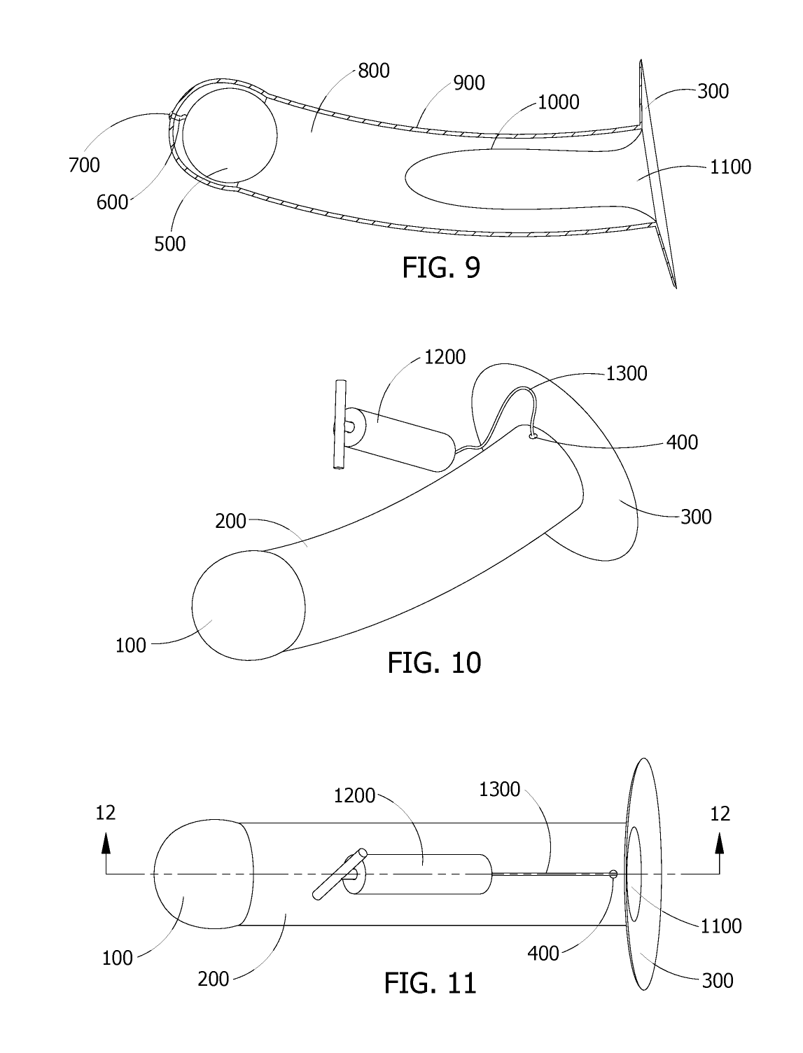

FIG. 9 is a cutaway side view of the invention of FIG. 2 with a bulb shaped pump located in the head. Shown in FIG. 9 are the: exterior wall 900; interior air bladder 800; flange 300; penile sheath 1000; penile port 1100; head tip outlet 700; pump connector 600; bulb pump 500; and, exterior wall 900.

FIG. 10 shows the invention of FIG. 1 with an external pump mechanism connected to the outlet towards the base. Shown in FIG. 10 are the: head 100; shaft 200; base tip outlet 400; flange 300; pump connector 600; piston pump 1200; and, pump connector 600.

FIG. 11 is a top down view of the configuration of FIG. 10. Shown in FIG. 11 are the: head 100; shaft 200; base tip outlet 400; flange 300; pump connector 600; penile port 1100; piston pump 1200; and, pump connector 600.

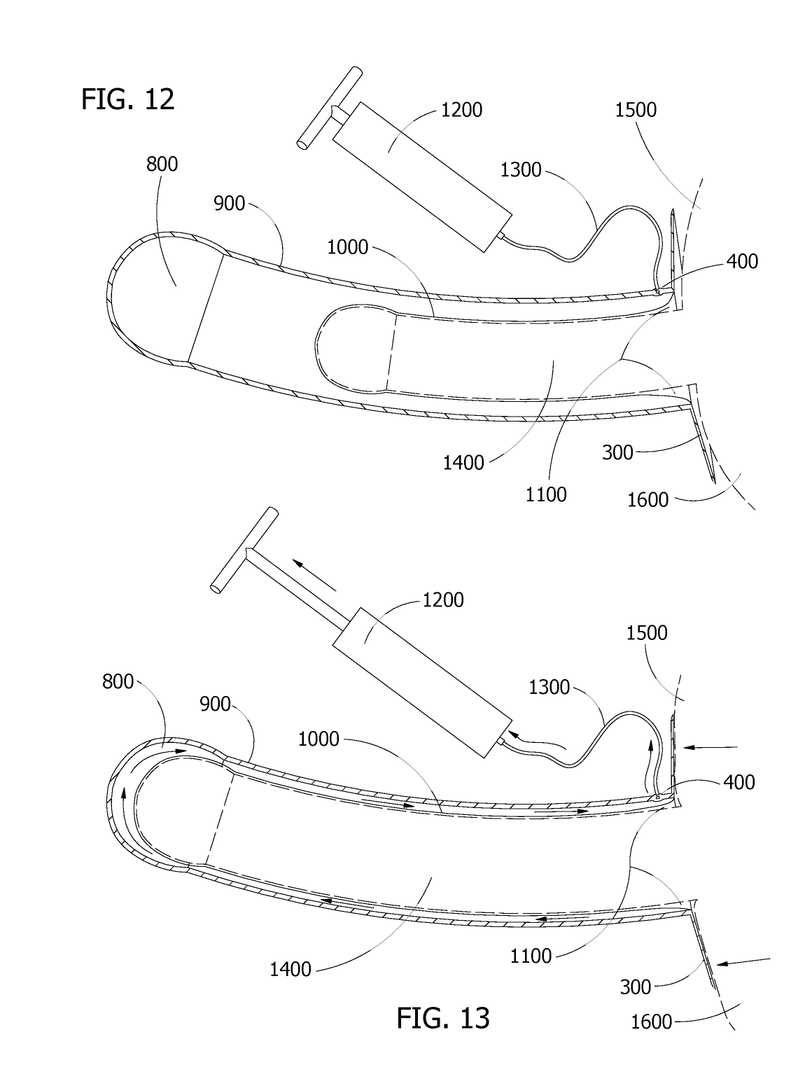

FIG. 12 is side view of the configuration of FIG. 10 invention as donned by a user with a soft penis. Shown in FIG. 12 are the: exterior wall 900; interior air bladder 800; flange 300; base tip outlet 400; penile sheath 1000; penile port 1100; pump connector 600; piston pump 1200; penis 1400; user abdomen 1500; and, scrotum 1600.

FIG. 13 is side view of the configuration of FIG. 10 invention as donned by a user after a vacuum pump has removed air from the internal air bladder. Shown in FIG. 13 are the: exterior wall 900; interior air bladder 800; flange 300; base tip outlet 400; penile sheath 1000; penile port 1100; pump connector 600; piston pump 1200; penis 1400; user abdomen 1500; and, scrotum 1600.

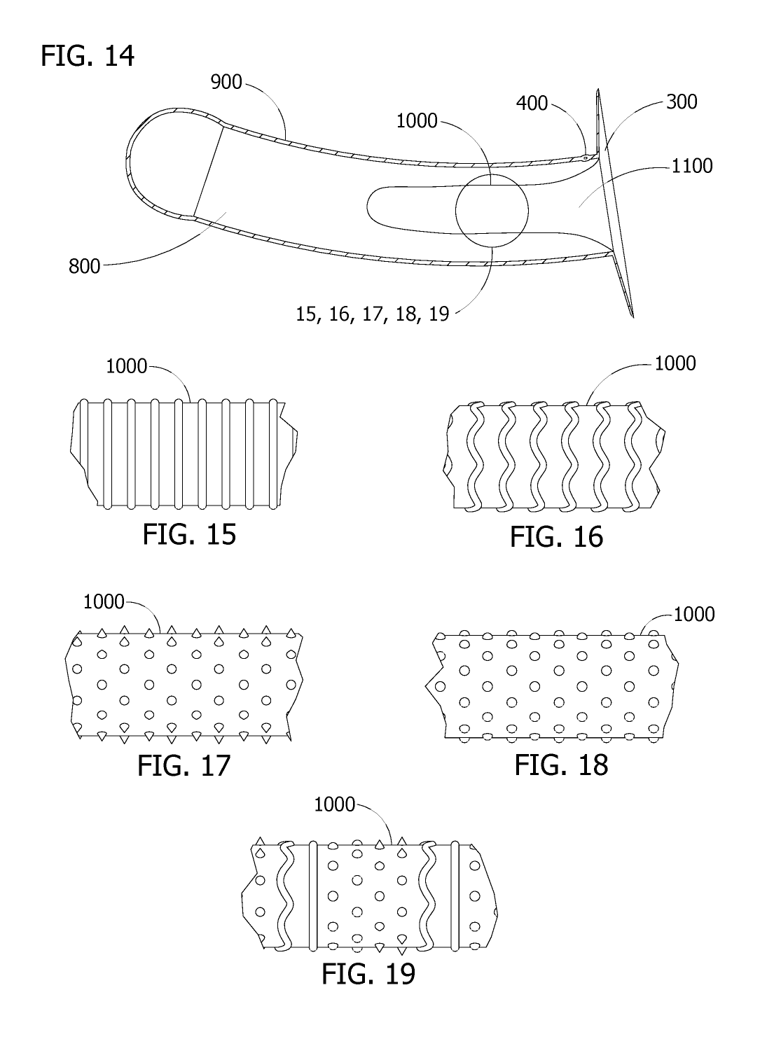

FIG. 14 is side view of the invention centering focus on the texture of the penile sheath. Shown in FIG. 13 are the: exterior wall 900; interior air bladder 800; flange 300; base tip outlet 400; penile sheath 1000; and, penile port 1100. Focus is drawn to some of the potential textures of the penile sheath as further shown and described in FIGS. 15-19.

FIG. 15 is an example of one of many ribbing textures that are applied to the interior of the penile chamber.

FIG. 16 is an example of another ribbing texture applied to the interior of the penile chamber.

FIG. 17 is an example of a spiked texture applied to the interior of the penile chamber.

FIG. 18 is an example of a bumped texture applied to the interior of the penile chamber.

FIG. 19 is an example of a multi treaded form of texture applied to the interior of the penile chamber.

Although a harness strap, saddle, perineum massager, may be added to the invention, the invention avoids the need for many of these extra features.

Although the device can be used by persons with erectile dysfunction and/or suffering from premature ejaculation, it is also adapted to accommodate an erect penis so that a person with normal or increased sexual stamina and energy can use the device with an erect penis.

After climax, if the penis softens or becomes less erect, the donning user can continue to pleasure their sexual partner. This is an encore method of intercourse. The sexual aid device may be utilized before or after climax. The flange 300 enables the device when to be easily donned by a person with a flaccid penis as it is placed over the flaccid penis and as the vacuum removes air from the chamber the flaccid penis expands inside the device, much like a VCD. Although the figures show the use of bulb and piston types of pumps nearly any appropriately sized pump could be used including motorized and spring powered pumps, air can also be drawn out through a straw attached to a vacuum, human mouth, or other suction device. In an embodiment, connectively associate with the outlet, pump and interior air bladder is a close and release valve that enables the donning user or their partner to fix and release the air pressure within the vacuum. Incorporating the release valve into the pump mechanism via switch, button, bulb etc. enables the operator to adjust the pressure of the vacuum on the fly. However, there release valve may be incorporated into a separate outlet/inlet.

In a preferred embodiment, the outer shell of sexual aid device and system of the present invention is made of a single material from a single mold. The material used for a sexual aid device and system of the present invention may be nearly any solid material. However, a material used often has elastic properties in order to absorb energy and stretch to fit. In a preferred embodiment a sexual aid device and system of the present invention fits around a penis and is held in place by suction on the penis and at the base of the penis through the flange. This preferred embodiment provides an added benefit of enabling the automatic massaging, squeezing and pulling sensation around the penis that provides pleasure to the penetrating partner donning the device, similar to that of normal sex with penetration. For the penetrated/receiving partner the device provide the sensation of a solid erect penis, when the penetrating partner would normally be flaccid or unable to penetrate.

A preferred material for forming the outer shell of the sexual aid device and system of the present invention contains polysiloxanes, a silicone plastic and/or silicone rubber blend. Various elastomers may also be used, including but not limited to natural rubbers. Ideally, in many cases, an inert or medical grade silicone rubber, latex or silicone blended material is used. The recommended materials for forming a thrust bumper are biocompatible and hypoallergenic, but can also be treated with an antibiotic or antiviral coating and/or texture. Antibiotic material and/or chemicals may also be infused or formed within the material of the sexual aid device and system of the present invention. Springs, foams, padding, goose down, cotton, rayon, polyester, various microfibers and other materials such as memory foam may be used to form the sexual aid device and/or portions of its core. The chassis may use metals and other more rigid materials for structural support. However, it is desirable that the outer and inner surfaces are soft and hygienic.

The sexual aid device and system of the present invention core may contain gels, liquids and/or other materials. However, too much memory or too much elasticity of the material that comprise a sexual aid device and system of the present invention may adversely effect the results. Moreover, many springs, foams, padding and other materials often lack the generally desired hypoallergenic characteristics. The best materials generally are: highly elastic; quickly to return to their stable (molded or cut) form; resist/prevent and/or destroy bacteria, fungus and viruses; and, are easily washed. It is desirable that there is sufficient room inside the inner chamber so that the flaccid penis of the donning user is able to expand to and beyond its maximum erect length and girth. A preferred embodiment of a chassis of the present invention compresses during thrusting of sexual intercourse and expands during withdraw. Generally, the material forming a sexual aid device and system of the present invention should feel comfortable to the touch and soft, silky, and skin like. However, some users desire a more rigid, leathery, wet or rough feel. Ribbing, various textures such as surface spikes and treats are applied to some devices for additional sensation, pleasure and comfort.

The internal edges and outer edges of the sexual aid device are preferably smooth and beveled/rounded. Preferably, the edges are eased, but bullnose, ogee, straight and other forms of edge may be used. The rounded edges generally provide greater comfort and make it easier for a user to don the sexual aid device and prevent chaffing.

A preferred material for forming the exterior wall 900 and flange 300 of the sexual aid device and system of the present invention are generally those containing polysiloxanes, a silicone plastic and/or silicone rubber blend. Various elastomers may also be used, including but not limited to natural rubbers. Ideally, in many cases, an inert or medical grade silicone rubber, latex or silicone blended material is used. The recommended materials for forming the sheath are biocompatible and hypoallergenic, but can also be treated with an antibiotic or antiviral coating and/or texture. Antibiotic material and/or chemicals may also be infused or formed within the material of the sexual aid device and system of the present invention. Thin and disposable sheaths are desirable in many instances and materials such as lambskin or condoms are used. Spermicidal, antibiotic, lubricants, warming gels, cooling gels and antiviral coatings can enhance the sexual experience and prophylactic use of the device. Various textures, ribbing and treads patterns can be applied to the inner and outer surfaces of the sheath for greater sensation and control by both the donning user and the receiving person.

Beveling and grooving/texturing of the exterior edges also provides a comfortable fit while enabling better grip for donning and removing the device. Examples of various embodiments of external edges and groves are shown in the figures.

In order to don a sexual aid device, the giving and/or penetrating partner places his/her phallus, appendage and/or penis 1400 through the opening in the flange 300 of the device. Then air is removed from the interior air bladder 800 causing the penile sheath 1000 to expand toward the exterior wall 900. This creates suction on the penis 1400 and between the flange and the abdomen 1500, scrotum 1600 and/or pelvis.

In some embodiments the penile port 1100 is narrowed in order to provide erection ring type support for longer sex and greater engorgement of the penis. This is particularly useful when the donning/penetrating partner desires the sensation of an erection but suffers from erectile dysfunction, premature ejaculation, and/or is recovering from prior climax.

While results vary slightly across reputable studies, the general consensus is that the mean human penis, when erect, is in the range 12.9-15 cm (5.1-5.9 in) in length. The average girth is about four and fifty nine one-hundredths (4.59) inches in circumference. Consequently, the desired length of the penile channel from the proximal end of the penile port 1100 to the distal end should generally be greater than six (6) inches. In most applications, seven (7) inches or more is desired. The penile port 1100 circumference generally should be at least approximately one and one-half (1.5) inches and generally less than seven (7) inches. In most applications, between four (4) to six (6) inches of circumference is desired. The most common width being approximately one and one-half (1.5) inches or just under five (5) inches in circumference. However, some penile channel 4100 and penile port 2400 shapes and size may exceed or fall outside the generally recommended rages. In particular, in some applications it is desired to contain a vibrating mechanism(s), such as a unbalanced motor(s), spring(s) or bells. In these embodiments that chassis are often extended to include such mechanisms, and such mechanisms are activated prior and/or during insertion of the sexual aid device into an orifice. The drawings herein are understood to be just representative embodiments, other embodiments and combinations are also described herein.

Having thus described preferred embodiments, it should be apparent to those skilled in the art that certain advantages of the described system have been achieved. It should also be appreciated that various modifications, adaptions, and alternative embodiments thereof may be made within the scope and spirit of the present invention. The invention is further defined by the following claims.

What is claimed is:

1. A sexual aid device comprising:
an outer wall;
an interior air bladder;
an outlet from the interior air bladder;
a penile port;
a penile sheath;
a pump;
wherein the pump is configured to remove air from the interior air bladder and cause the penile sheath to expand toward the outer wall.

2. The sexual aid device of claim 1 further comprising a flange.

3. The sexual aid device of claim 1 further comprising a bulb shaped pump.

4. The sexual aid device of claim 1 further comprising a head wherein the pump is incorporated into the head.

5. The sexual aid device of claim 1 further comprising a head wherein the head comprises a head tip and an outlet.

6. The sexual aid device of claim 2 wherein the pump is incorporated within the flange.

7. The sexual aid device of claim 4 further comprising a bulb shaped pump.

8. The sexual aid device of claim 5 further comprising a head wherein the outlet is configured to selectively retain and release air from the interior air bladder.

9. The sexual aid device of claim 6 further comprising a bulb shaped pump.

10. The sexual aid device of claim 6 further comprising an outlet wherein the outlet is configured to selectively retain and release air from the interior air bladder.

11. The sexual aid device of claim 7 further comprising an outlet wherein the outlet is configured to selectively retain and release air from the interior air bladder.

12. The sexual aid device of claim 10 further comprising a close and release valve

13. The sexual aid device of claim 11 further comprising a close and release valve.

14. The sexual aid device of claim 12 wherein the close and release valve is operated in the same place as the pump.

15. The sexual aid device of claim 13 wherein the close and release valve is operated in the same place as the pump.

16. The sexual aid device of claim 14 further comprising a base shaft incorporating a tension ring at the base of the shaft.

17. A system of sexual aid devices used for sexual intercourse comprising:
a sexual aid device comprising:
an outer wall;
an interior air bladder;
an outlet from the interior air bladder;
a penile port;
a penile sheath;
an outlet for connection to external pump;
and,
an external pump configured to vacuum air from the device through the outlet for connection to external pump.

18. The system of claim 17 further comprising a tension ring.

19. The system of claim 18 wherein the tension ring is incorporated into a shaft surrounding the penile port.

20. A method of sexual intercourse comprising:
donning with a flaccid penis:
a sexual aid device comprising:
an outer wall;
an interior air bladder;
an outlet from the interior air bladder;
a penile port;
a penile sheath;
a pump;
wherein the pump is configured to remove air from the interior air bladder and cause the penile sheath to expand toward the outer wall.
